(19) United States
(12) Patent Application Publication
(10) Pub. No.: US 2021/0208134 A1
(43) Pub. Date: Jul. 8, 2021

(54) SYSTEMS AND METHODS OF OPTICALLY DETERMINING ANALYTE CONCENTRATIONS

(71) Applicant: Louay Khaled Fakhro, Mission Viejo, CA (US)

(72) Inventor: Louay Khaled Fakhro, Mission Viejo, CA (US)

(21) Appl. No.: 17/133,317

(22) Filed: Dec. 23, 2020

Related U.S. Application Data
(60) Provisional application No. 62/956,717, filed on Jan. 3, 2020.

(51) Int. Cl. G01N 33/52; G01N 33/68; G01N 33/487; G01N 33/49

(52) U.S. Cl. CPC G01N 33/52; G01N 33/49; G01N 33/48792; G01N 33/68

(57) ABSTRACT

Systems, devices, and methods directed to rapidly, dynamically, and intelligently estimating analyte data from a lateral flow test strip are disclosed. Systems of the inventive subject matter feature a testing device and a test strip, where a biological sample is applied to the test strip and that test strip is then inserted into a corresponding testing device for analysis. A variety of analyte concentrations in blood can be measured using systems and methods of the inventive subject matter, including various amino acids such as phenylalanine. Systems and methods of the inventive subject matter facilitate rapid, at-home testing with result times a fraction of what has previously been possible.

This application claims priority to U.S. Provisional Patent Application Ser. No. 62/956,717, filed Jan. 3, 2020. All extrinsic materials identified in this application are incorporated by reference in their entirety.

### FIELD OF THE INVENTION

The field of the invention is optical testing to determine concentrations of amino acids in liquid biological samples.

### BACKGROUND

The background description includes information that may be useful in understanding the present invention. It is not an admission that any of the information provided in this application is prior art or relevant to the presently claimed invention, or that any publication specifically or implicitly referenced is prior art.

Phenylketonuria (PKU) is an autosomal recessive disorder in which the enzyme phenylalanine hydroxylase (PAH) is non-functional in its conversion of phenylalanine to tyrosine, and if this disorder is poorly managed, there will be deficient growth, microcephaly, seizures, intellectual impairment, and physical degradation. PKU management should start as soon as possible after diagnosis and be maintained throughout life to prevent irreversible damage. Currently, the best treatment practices involve strict diet control and regular monitoring of phenylalanine concentrations in the blood to guide dietary decisions. But regular monitoring of phenylalanine concentration in the blood is not always good enough as blood testing can involve long turn-around times leading to information that a week old or older. Since phenylalanine changes can occur within two hours after a meal, the current state of the art leaves people suffering from PKU at risk.

Thus, using current technologies, medical practitioners and their patients have no choice but to use data that is seven days old (or older) when the data being measured fluctuates moment-to-moment based on a variety of different physiological processes (e.g., eating). This flawed treatment plan ultimately leads to an annual economical burden for each patient estimated between $15,000 and $200,000 driven by the severity level of the disease and economic ability of caregivers and families to support dietary guidance in the early stages of the disease. Costly testing, hospitalization, and insurance claims are avoidable if patients are empowered to take a more active role in monitoring their own blood phenylalanine concentrations, enabling better informed day-to-day—and even hour-to-hour—dietary decision making.

The current state of the art features tests performed using liquid chromatography tandem mass spectrometry (LC-MS/MS), which involves expensive and multi-purpose machines designed to measure many different analytes in addition to phenylalanine. Some attempts have been made to rapidly monitor phenylalanine levels in individuals with PKU. These attempts have required chemical processes that demonstrate feasibility within a laboratory setting. But while the use of laboratory grade processes show promise, these alternatives cannot be easily incorporated into simple to use, at-home devices, and misguided approaches to these techniques have resulted in poor measurement accuracy caused by lot variations of enzymes, instabilities, and in some cases hematocrit interference.

It has yet to be appreciated that systems and methods of testing that facilitate at-home tests that generate rapid, accurate results are possible. Thus, there is still a need in the art for improved testing devices with accompanying test strips to measure and monitor concentrations of various analytes in the blood.

### SUMMARY OF THE INVENTION

The present invention provides apparatuses, systems, and methods directed to detecting amino acid concentrations in liquid biological samples. In one aspect of the inventive subject matter, an analyte measurement system is contemplated as comprising: a test strip having a handling portion, a liquid sample insertion site, a lateral flow pathway connected to the liquid sample insertion site, the lateral flow pathway comprising a plasma separation membrane, and an optically transparent measurement window coupled with the lateral flow pathway, where the optically transparent measurement window includes a reactant configured to create a dye when it reacts with a at least a portion of a liquid sample. The system additionally includes a testing device, comprising: a cartridge port configured to receive the test strip; a light source configured to emit light toward a photon sensor such that the optically transparent measurement window is disposed between the light source and the photon sensor when the test strip is inserted into the cartridge port; an analog-to-digital converter (ADC) configured to convert an analog signal from the photon sensor into a digital signal; and a microcontroller configured to determine an analyte concentration based on the digital signal.

In some embodiments, the reactant comprises a tetrazolium salt, and the analyte can be an amino acid such as phenylalanine. In some embodiments, the lateral flow pathway further comprises at least one of a nitrocellulose membrane, an asymmetric polysulfone, a hydrophilic glass fiber, and hydrophilic cotton linter materials. The liquid sample insertion site can include a second plasma separation membrane layer, a conjugate pad layer, and a nitrocellulose sub-layer. The test strip can additionally feature a slot configured to ensure the test strip is seated and positioned properly upon insertion into the testing device. In some embodiments, light sources in the testing device can emit light between 200 nm and 600 nm in wavelength.

In another aspect of the inventive subject matter, a liquid biological sample testing device is contemplated. The device includes: a cartridge port configured to receive a liquid biological sample test strip comprising an optically transparent measurement window; a light source configured to emit light toward a photon sensor such that the optically transparent measurement window is disposed between the light source and the photon sensor when the liquid biological sample test strip is inserted into the cartridge port; an analog-to-digital converter (ADC) configured to convert an analog signal from the photon sensor into a digital signal; and a microcontroller configured to determine an analyte concentration in a liquid biological sample disposed on the liquid biological sample test strip based on the digital signal.

In some embodiments, the testing device additionally includes at least one of a collimating optic, a short pass optical filter, a dichroic filter, and a cut-on optical filter. It is contemplated that light sources of the inventive subject matter can emit light between 200 nm and 600 nm in wavelength.

In another aspect of the inventive subject matter, a liquid biological sample test strip is contemplated. The test strip includes: a handling portion; a liquid sample insertion site; a lateral flow pathway connected to the liquid sample insertion site, the lateral flow pathway comprising a plasma separation membrane; an optically transparent measurement window coupled with the lateral flow pathway, where the optically transparent measurement window comprises a reactant configured to create a dye when it reacts with at least a portion of a liquid sample; and a slot that is configured to ensure the test strip is seated and positioned properly upon insertion into a testing device.

In some embodiments, the lateral flow pathway further comprises at least one of a nitrocellulose membrane, a hydrophilic glass fiber, and hydrophilic cotton linter materials, and it is contemplated that the reactant includers a tetrazolium salt. In some embodiments, the liquid sample insertion site comprises a second plasma separation membrane layer, an asymmetric polysulfone, a conjugate pad layer, and a nitrocellulose sub-layer. In some embodiments, the test strip additionally includes a slot that is configured to ensure the test strip is seated and positioned properly upon insertion into the testing device.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic showing modules of a testing device of the inventive subject matter.

FIG. 2 shows a testing device of the inventive subject matter.

FIG. 3 shows a cutaway view of a testing device of the inventive subject matter.

FIG. 4 shows a back view of the testing device of FIG. 2.

FIG. 5 shows a left-side view of the testing device of FIG. 2.

FIG. 6 shows a bottom view of the testing device of FIG. 2.

FIG. 7 shows a right-side view of the testing device of FIG. 2.

FIG. 8 shows a perspective view of the testing device of FIG. 2 with a base station.

FIG. 9 shows a test strip of the inventive subject matter.

FIG. 10 shows a cutaway view of a testing device with an accompanying test strip, showing a zoomed view with the test strip fully inserted.

FIG. 11 is a functional flowchart for testing devices of the inventive subject matter.

FIG. 12 is a flowchart showing optical module functions.

FIG. 13 is a flowchart showing how optical information is collected and processed.

FIG. 14 shows an example of the layers in a liquid sample insertion site of a test strip the inventive subject matter.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used in the description in this application and throughout the claims that follow, the meaning of “a,” “an,” and “the” includes plural reference unless the context clearly dictates otherwise. Also, as used in the description in this application, the meaning of “in” includes “in” and “on” unless the context clearly dictates otherwise.

Also, as used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth in this application should be interpreted as being inclusive of their endpoints and open-ended ranges should be interpreted to include only commercially practical values. Similarly, all lists of values should be considered as inclusive of intermediate values unless the context indicates the contrary.

It should be noted that any language directed to a computer should be read to include any suitable combination of computing devices, including servers, interfaces, systems, databases, agents, peers, Engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network. The following description includes information that may be useful in understanding the present invention. It is not an admission that any of the information provided in this application is prior art or relevant to the presently claimed invention, or that any publication specifically or implicitly referenced is prior art.

Systems of the inventive subject matter are designed to facilitate rapid and sensitive quantification of substances in the blood such as chemicals, amino acids, molecules, hormones, and the like. The system in this application is described in the context of detecting phenylalanine levels in the blood, but this should not be considered limiting. Thus, systems of the inventive subject matter include a testing device configured to receive test strips, where the device is capable of identifying presences and levels of different chemicals, hormones, amino acids, etc. that appear in a user's blood when applied to a test strip of the inventive subject matter. Systems of the inventive subject matter comprise two main devices, a testing device and a test strip. Testing devices are configured to analyze test strips upon inserting a test strip into the testing device, and test strips are configured such that users can apply a small amount of blood for analysis.

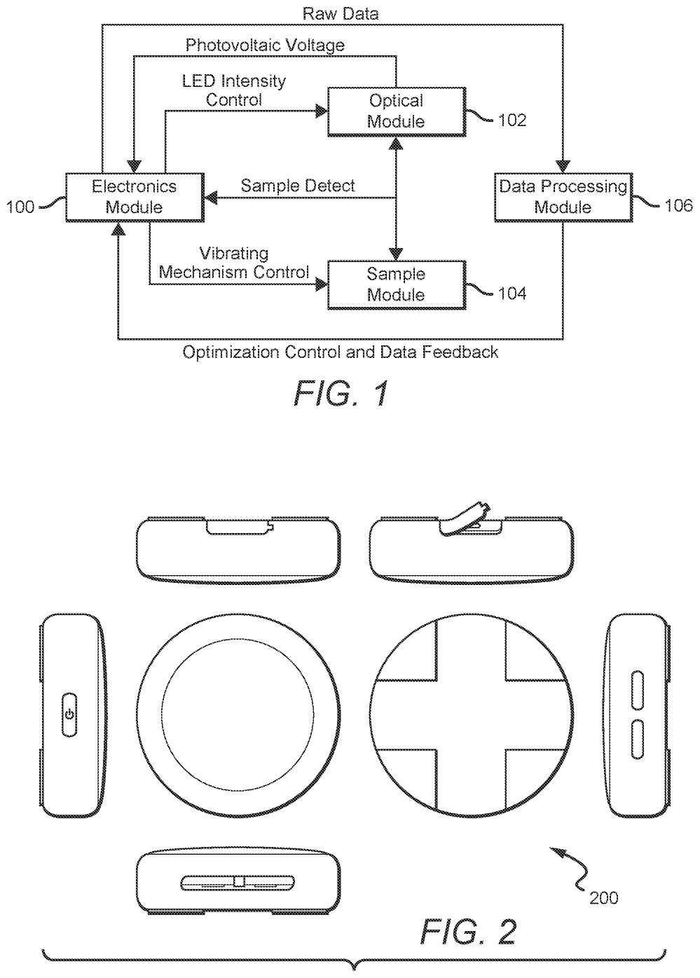

FIG. 1 is a schematic showing the different modules of a testing device, including an electronics module 100, an optical module 102, a sample module 104, and a data processing module 106. Electronics module 100 includes a microcontroller (MCU) and an analog-to-digital converter (ADC). Microcontrollers (MCUs) can be implemented in embodiments of the inventive subject matter. For example, MCUs made by Microchip, ST Microelectronics, On Semiconductor, Maxim Integrated, NXP, Renesas, Infineon, etc., and ADCs made by Microchip, Maxim, Texas Instruments, Analog Devices, etc., can be implemented. As one example, a printed circuit board containing a Microchip MCU with a separate Microchip ADC can be used. Electronics module 100 is configured to receive raw data from optical module 102 so it can convert that data into a form or format usable by the data processing module 106. Electronics module 100 is also configured to detect the presence of a test strip as well as to optionally detect test strip authenticity to prevent use of counterfeit test strips. Because test strips a separate components from testing devices of the inventive subject matter, test strips must be inserted into testing devices so the testing devices can analyze blood applied to those test strips.

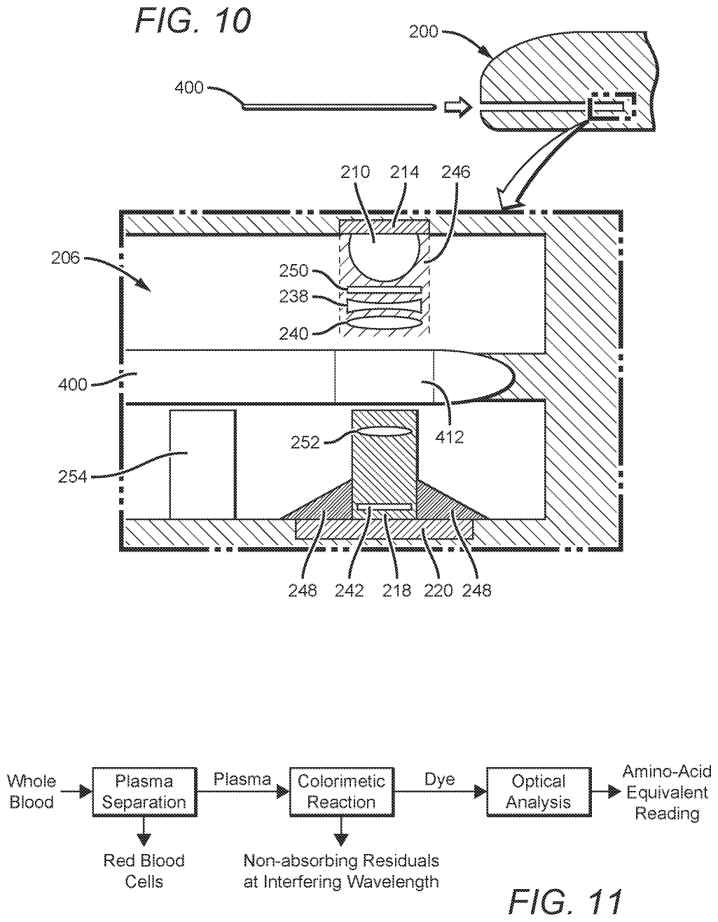

To improve analysis, electronics module 100 can additionally include a vibrating mechanism to encourage lateral blood flow on a test strip (e.g., faster flow than would ordinarily occur). Thus, once a test strip is inserted into a testing device of the inventive subject matter, electronics module 100 can activate a vibrating mechanism to encourage lateral blood flow on the inserted test strip, which helps blood move into portions of the test strip where optical analysis takes place. Lateral blood flow occurs in the absence of vibration, and the vibrating mechanism can be implemented to encourage more rapid blood flow. Vibrating mechanisms contemplated include rotating weight vibrating mechanisms (e.g., an off-center weight affixed to a rotor) and piezoelectric vibrating mechanisms (or both). Rotating weight vibrating mechanisms can encourage plasma separation while piezoelectric vibrating mechanisms can increase plasma mobility and reaction speeds. Example vibrating mechanism 254 is shown in FIG. 10.

Optical module 102, which is also shown in FIG. 10 and explained in more detail below, includes a light source (e.g., an LED, a laser diode). Light sources can be configured to emit radiation between 200 nm and 600 nm in wavelength, preferably around 450 nm. Other wavelengths are also contemplated, for example, wavelengths between 220 nm and 4000 nm are contemplated and can be useful depending on the analyte of interest. Wavelength can be selected based on absorbing characteristics of a biological sample to be analyzed. In some embodiments, optical module 102 also includes one or any combination of one or more collimating optics, one or more short pass optical filters, one or more dichroic filters, and one or more cut-on optical filters.

Sample module 104 includes a security chip to allow for authentication. Once a test strip is inserted into a testing device of the inventive subject matter, sample module 104 can determine whether that test strip is authorized for use with the testing device by using its security chip to read information from the test strip. This prevents the use of test strips not intended for use with the testing device that, e.g., may be counterfeit or otherwise unable to produce accurate, reliable results Data processing module 106 is a software-based module run by, e.g., a microcontroller (or, in some embodiments, a microprocessor) included in electronics module 100, and it is configured to interpret results of analysis after those results have been converted to a digital signal by electronics module 100.

FIG. 2 shows a testing device 200 of the inventive subject matter from a variety of angles include top, bottom, front, back, left, and right, with an additional view showing an I/O port (e.g., a USB port of any type or protocol). Some of these views are discussed in more detail below to describe the different features they demonstrate.

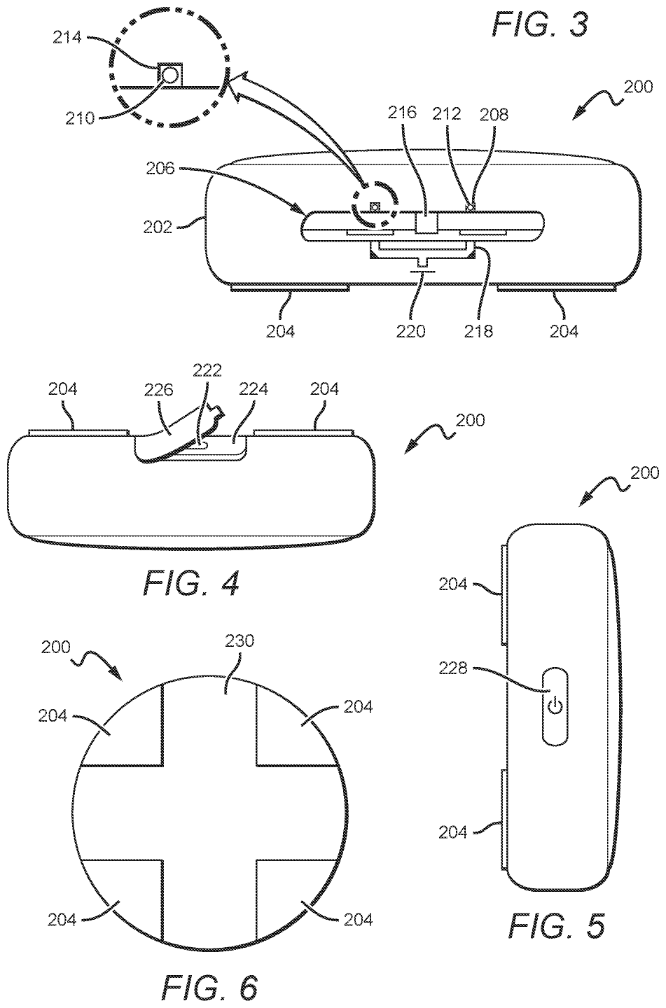

FIG. 3 shows a cutaway view of testing device 200, making many of its internal component visible. It includes an outer shell 202 with friction pads 204 attached to a bottom surface of outer shell 202. Friction pads 204 can be made from, e.g., rubber, metals, ceramics alloys, composites, plastics, etc. Cartridge port 206 is configured to receive test strips. Device 200 includes a first light source 208 and a second light source 210. The first light source 208 is coupled with a first temperature controlled light source fixture 212 and a second temperature controlled light source fixture 214. First light source 208 and second light source 210 can comprise one or more LEDs, laser diodes, or any other light source such as a halogen bulb, a xenon bulb, fiber optic element, or any other type of light source configured to emit radiation within a desire range of wavelengths. First temperature controlled light source fixture 208 and second temperature controlled light source 210 are contemplated to be electronically controlled. Alternative embodiments of the inventive subject matter can include just a single light source with all other aspects of the testing device and, in some cases, the test strip being modified accordingly. In some embodiments, additional temperature controllers can be implemented to ensure proper operating temperatures are maintained. It should be understood that although this application discusses the use of two light sources, it should be understood that any number of additional light sources can be implemented into embodiments of the inventive subject matter. With an increase in number of light sources, components and subsystems that support the functions of each added light source are also included.

When second light source 210 is activated, it shines light toward light transferring component 218, e.g., a light pipe, which contains collimating optic 242 (where, e.g., collimating optic 242 can comprise a cut-on filter) and transports light to photon sensor 220. Additional details regarding the optical module are described regarding FIG. 10 below. Light transferring components 218 and 246 can comprise, e.g., fiber optics (such as fiber optic lines made from various glasses or silica materials such as borosilicate glass and including other types of non-silica glasses such as fluoride glass, phosphate glass, etc.). Light transferring components of the inventive subject matter can also be as basic as optical acrylics or polycarbonate materials and can be formed into, e.g., a solid cylinder that has an optically transmissive center. In some embodiments, light transferring components can be formed into hollow tubes or cutouts from the housing material that simply create an optical pathway. Testing device 200 additionally features a latching interface guide rail 216 (shown in FIG. 3) that is configured to ensure that a test strip, upon insertion into device 200, seats properly for optical analysis.

Upon activation while a test strip is inserted, first light source 208 and second light source 210 direct light toward regions of the test strip designated for optical analysis. Light passes through light conditioning components (e.g., 250, 238, and 240), e.g., within 246 before passing through optically transparent measurement windows 412 and 414 on the inserted test strip (see FIG. 9 for further description of a test strip) before passing into light transferring component 218 and on to photon sensor 220. Photon sensor 220 converts light to measurable voltage, which is discussed in more detail below regarding FIG. 10.

FIG. 4 shows a side view of testing device 200, showing port 222 that can be used for charging/recharging and other input/output tasks. In some embodiments, port 222 can feature a serial communication port such as USB-A, USB-B, USB-C or any other serial port and including any serial communication protocol (e.g., USB 2.0, 3.0, etc.). Port 222 can be inset within a recessed area 224 on testing device 200, and port 222 can be covered by flexible cover 226, which covers port 222 and can also cover all or any portion of recessed area 224. Cover 226 can be made from, e.g., a rubber material and be configured to prevent debris or fluids from getting into port 222.

Port 222 can be, for example, a port to receive a serial connection (e.g., any USB protocol) that can also be configured to supply power to the device, for example, to charge an on-board battery. In addition to charging, port 222 can be used to transfer data to and from testing device 200. Thus, after performing analysis on a blood sample present on a test strip, information including the results of that analysis can be transferred to another device via port 222. In some embodiments, test device 200 can include a wireless communication module to facilitate wireless data transfer. A wireless communication module can be used to allow testing devices of the inventive subject matter to directly upload test results and other gathered data to the internet (e.g., to server storage for later access by a user or by authorized healthcare professionals). A wireless communication module can comprise one or any combination of, e.g., near field communication (NFC), Bluetooth low energy (BLE), or similar wireless protocols to transfer data to, e.g., a local smart device such as a phone having specialized software. In some embodiments, one or any combination of a WiFi, cellular, Sub-GHz, or the like can be used to transfer data to a remote server (e.g., a cloud service) either via mobile computing device (e.g., a smart phone) or via a local WiFi router or internet hotspot.

FIG. 5 shows another side view of testing device 200, showing an input button 228 that can be used to power the device on and off. In some embodiments, input button 228 can additionally be used for input while the device is powered on (e.g., to make a menu selection or the like). This can be accomplished by, e.g., interpreting a long presses for power on/off and short presses for other inputs. Additional buttons can be added without deviating from the inventive subject matter.

FIG. 6 shows a bottom view of testing device 200. Beneath bottom surface 230 of testing device 200, wireless charging components can be included. For example, a charging coil compatible with the Qi wireless charging standard can be incorporated such that testing device 200 can be charged wirelessly. In some embodiments, bottom surface 230 comprises an electromagnetic coupling receiver pad. Any wireless charging protocol can be implemented in embodiments of the inventive subject matter.

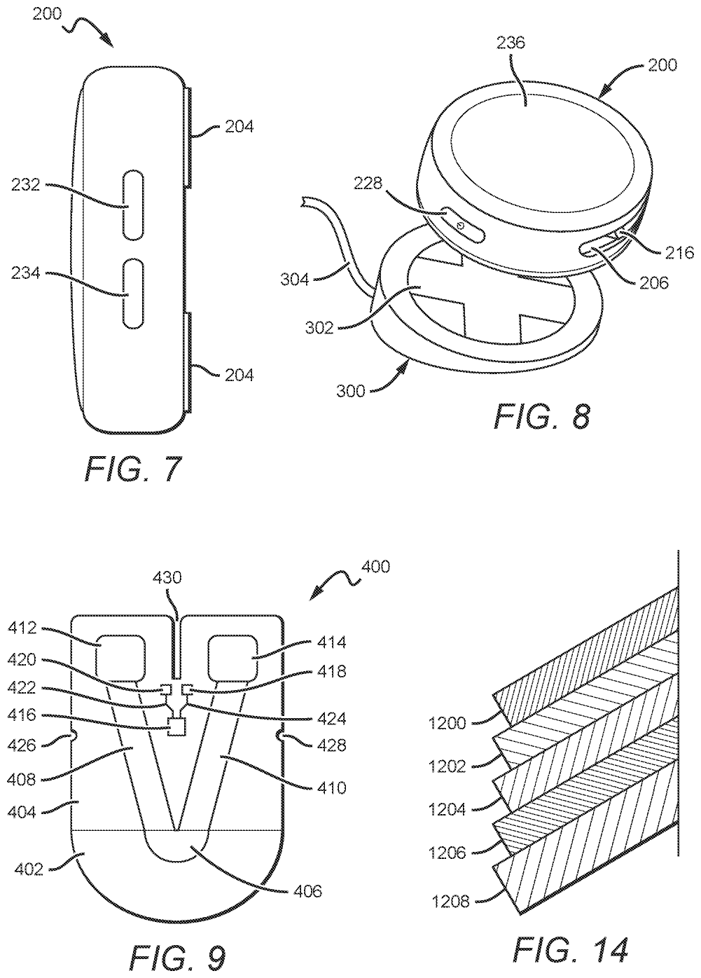

FIG. 7 shows another side view of testing device 200, showing first input 232 and second input 234. First input 232 and second input 234 can be configured to allow a user to provide input into testing device 200. It is contemplated that all of inputs 228, 232, and 234 can be configured as physical buttons, touch-based buttons, or any combination thereof. Touch-based buttons can implement, e.g., capacitive or resistive touch technologies to help avoid buildup of dust in spaces created by physical buttons.

FIG. 8 shows testing device 200 along with a corresponding base station 300. Base station 300 can be configured such that testing device 200 rests on its top surface. In some embodiments, base station 300 features wireless charging components to facilitate charging testing device 200. Wireless charging components can be incorporated below top surface 302, and, in some embodiments, top surface 302 can incorporate an electromagnetic coupling transmitting pad. Base station 300 can additionally include a cable 304 that can plug into a power source. This view of testing device 200 also shows display 236. Display 236 can be used to show users information such as testing results, battery level, menu options, and so on. In some embodiments, display 236 comprises a touch-based display to allow for a broader range of user input. Base stations of the inventive subject matter can also include wireless communications hardware to facilitate network communication with, e.g., cloud servers and the like. Base stations of the inventive subject matter can also be configured to use a nearby smartphone, wireless router, or cell network to connect to the Internet, and they can additionally or alternatively feature their own integrated hardware to facilitate direct Internet connectivity (e.g., via cellular network connection).

FIG. 9 shows a test strip 400 of the inventive subject matter. Test strip 400 has a handling portion 402 and a testing portion 404. Handling portion 402 is intended to allow users to hold test strip 400 without touching any of its sensitive areas (e.g., areas where biological samples such as blood are deposited) that could impact analysis upon inserting test strip 400 into a testing device of the inventive subject matter (e.g., testing device 200), though even sensitive areas are typically protected by outer layers such that a biological sample is deposited to a substrate beneath such an outer layer. Test strip 400 includes a liquid sample insertion site 406. Liquid sample insertion site 406 is configured to receive a biological sample such as blood. In some embodiments, other biological fluids (e.g., saliva, urine, plasma, etc.) can be applied to the liquid sample insertion site 406.

Once a biological sample is applied to liquid sample insertion site 406, it travels up the left lateral flow pathway 408 and the right lateral flow pathway 410. One side can be configured to act as a control to capture a baseline condition of free nicotinamide adenine dinucleotide (NADH) particles so that upon measurement of the opposite side, excess NADH can be cancelled out. To improve biological fluid flow along each of the lateral pathways, lateral pathways 408 and 410 can comprise reference chemical reactants (e.g., surfactants such as non-ionic detergents “Tween 20” or “Triton X-100).

After a biological fluid is deposited onto liquid sample insertion site 406, it flows from biological sample insertion site 406 to traverse the lateral flow pathways 408 and 410 before it is deposited onto a left optically transparent measurement window 412 from the left lateral flow pathway 408 and onto a right optically transparent measurement window 414 from the right lateral flow pathway 410. By the time a biological fluid has reached each of the optically transparent measurement windows, it has been adequately mixed with chemicals to enable optical quantification at each site. Biological fluids generally flow according to capillary action.

In the case of using a blood sample, to avoid optical interferences caused by hematocrit within the blood sample, the liquid sample insertion site 406 where blood is applied can feature a plasma separation membrane in which surface tension between a blood sample and the membrane pulls blood through the pores of the material. In some embodiments, the plasma separation membrane is designed to have decreasing pore size as a fluid sample passes through the thickness of the material. Membrane pores that decrease in size across the membrane's thickness behave like capillary tubes, taking advantage of the phenomenon where adhesive forces between a liquid and a solid tube through which it passes are stronger than the cohesive forces between individual molecules within the liquid, thus allowing surface tension to dictate how far the liquid will travel. When, e.g., whole blood comes in contact with a membrane of the inventive subject matter, surface tension between the blood and the membrane encourages blood flow through the material. Decreasing pore size through the membrane allows separation to occur by taking advantage of size differences between constituents of whole blood. For example, red and white blood cells (6-8 μm and 12-17 μm in diameter, respectively) can be captured while plasma continues to flow through the membrane towards the reaction sites. To facilitate improved liquid mobility through the membrane and thereby reduce the time it takes for amino acid (e.g., phenylalanine) quantification, a vibrating or acoustic mechanism can be implemented.

After separation, plasma travels down both lateral flow pathways 408 and 410 towards the left and right optically transparent measurement windows 412 & 414 where the plasma forms an optically obstructive dye in proportion to the concentration of, e.g., phenylalanine (in, for example, units of mg/dL or μmol/L). In some embodiments, each measurement of amino acid concentration is sensitive to changes of less than 1 mg/dL. In some embodiments, microfluidics can additionally or alternatively be used to separate plasma from the blood instead of a membrane solution.

In the example of phenylalanine, two reactions can be used to colorimetrically quantify phenylalanine; the first reaction is an enzymatic reaction of phenylalanine dehydrogenase (PheDH) to catalyze a reaction between phenylalanine and an oxidized form of nicotinamide adenine dinucleotide (NAD+) into the reduced form of nicotinamide adenine dinucleotide (NADH), phenylalanine pyruvate (PhePyr), and ammonium ions (NH4+). The second reaction can use the NADH from the first reaction to react with a tetrazolium salt and an electron mediator to create NAD+ along with a formazan dye product. The formazan dye product intensity will be in proportion to NADH, which is also directly proportional to phenylalanine.

Thus, systems of the inventive subject matter indirectly measure concentrations of phenylalanine. When light shines through an optically transparent measurement window, the system measures how much light dims as it passes through that window (e.g., as that light has been absorbed). How much light dims as it passes through a measurement window is proportional with the concentration of a target analyte. Thus, a mathematical relationship can be developed that links light dimming with analyte concentration. Once a biological sample reaches the optically transparent measurement windows 412 and 414, dye is generated in proportion to NADH, which is in turn proportional to phenylalanine. Therefore, a phenylalanine concentration can be determined by optically measuring an amount of light that is absorbed by the dye that forms in the optically transparent measurement windows.

Lateral flow pathways 408 and 410 can be made from, in whole or in part (e.g., by layer), a plasma separation membrane (e.g., Vivid™ Plasma Separation Membrane from Pall Corporation). Other layers of the lateral flow pathways can comprise one or any combination of nitrocellulose membranes, hydrophilic glass fibers, and hydrophilic cotton linter materials (e.g., Whatman Standard 17 glass fiber from Cytiva, Fusion 5 single layer matrix membrane from Cytiva, and Whatman CF4 dipstick pad and papers from Cytiva)

Optically transparent measurement windows 412 and 414 can be made from material like glass or plastic (e.g., 6FDA-DAD, BPDA-3F, or similar plastic films) that encase a biological sample and hold it within the test strip while allowing for a very minimal optical measurement losses. Careful selection of material for the optically transparent measurement windows enables testing devices of the inventive subject matter to use LEDs having low power requirements.

Test strip 400 additionally includes a memory device 416. Memory device 416 can include in its storage a unique electronic identifier to facilitate coupling analytical results with test strip 400. Memory device 416 is coupled with a first conductive pad 418 and a second conductive pad 420 by two conductive lines 422 and 424. Conductive pads 418 and 420 are configured to for leads inside a testing device of the inventive subject matter to couple with the contacts, and once that electronic coupling is accomplished, information from memory device 416, including a unique identifier, is read into the testing device and correlated with data gathered and generated by the testing device.

Memory device 416 can additionally include a cryptographic device with internal memory storage capabilities. A cryptographic device can store a private key and be configured to generate a corresponding public key to facilitate encrypted communication between a testing device and a test strip, which can in turn be used to ensure validity of the test strip's origin. Both hardware and software-based encryption are contemplated.

To facilitate inserting test strip 400 with a testing device, test strip 400 features a left alignment notch 426 as well as a right alignment notch 428. Alignment notches 426 and 428 are configured to mate with features of a testing device that are sized, dimensioned, and positioned to allow the test strip to snap into a desired position inside the testing device. Test strip 400 additionally includes slot 430 that mates with the latching interface guide rail of a testing device, which also helps to ensure test strip 400 is seated and positioned properly upon insertion.

FIG. 10 shows how test strip 400 can be inserted into testing device 200 such that the testing device's optical module can analyze a biological fluid sample deposited on test strip 400. The inset image shows a zoomed in view of an optical module of testing device 200 when test strip 400 is fully inserted. Note that FIG. 10 shows a cutaway view of just one side of a test strip within a testing device. The opposite side is symmetrically configured as shown in, e.g., FIGS. 3 and 9.

Focusing on the inset image, which is a cutaway view, test strip 400 is shown fully seated within cartridge port 206. Optically transparent measurement window 412 on test strip 400 is thus positioned between second light source 210, collimating optic 250, short pass filter 238, and dichroic filter 240 and light transferring component 218, which comprises cut-on filter 252 and second collimating optic 242 and that directs light to photon sensor 220.

Upon activating second light source 210, light emitted from second light source 210 shines through light transferring component 246, first collimating optic 250, short pass filter 238, and through dichroic filter 240 before passing through optically transparent measurement window 412 where a biological sample can be deposited (e.g., where a biological sample travels after depositing it to a testing strip's liquid sample insertion site) for analysis. Once it has passed through optically transparent measurement window 412, light moves into light transferring component 218 where it passes through cut-on filter 252 and collimating optic 242 before it reaches photon sensor 220. a biological sample under test that is deposited onto the optically transparent measurement window 412 can then be analyzed.

Light source 210 can be, e.g., an LED or a laser diode. It is desirable that light source 210 emit light of, or in the range of, certain wavelengths, including between 200 nm and 600 nm, which is on lower side of UV-VIS spectrum. For example, in some embodiments light source 210 is configured to emit light having a 450 nm wavelength.

Collimating optic 250 is configured to condense light (e.g., collimate it) emitted by light source into a largely uniform beam to direct that light to a sample under test that is deposited to optically transparent measurement window 412. Collimating optic 242 is similarly configured to condense electromagnetic radiation that has passed through the sample under test deposited to optically transparent measurement window 412 and through cut-on filter 252 into largely a uniform beam directed toward photon sensor 220. Testing device 200 can additionally include light blockers 248 that are configured to reduce the amount of ambient light that can reach photon sensor 220. They can be made from any opaque material or a material that is otherwise configured to block electromagnetic radiation of certain wavelengths. The goal of both the collimating optic 242 and the light blockers 248 is to improve a signal-to-noise ratio of light reaching the photon sensor 220 and to improve linearity of the system overall by minimizing stray sources of light from interfering with the photon sensor 220 (e.g., a photodiode). A stray source of light can be, for example, light that otherwise reaching photon sensor 220 that was not initially emitted by second light source 210 or light from the light source that reaches photon sensor 220 without first passing through one or more of the various light affecting components (e.g., cut-on filter, collimator, etc.). Neither collimating optic need be achromatic, but in some embodiments, one or both can be achromatic.

Short pass filter 238 allows for certain wavelengths of light to pass while blocking others. In testing device 200, for example, 450 nm wavelength light can be allowed to pass while preventing higher wavelengths, such as 500 nm and higher, from passing through. Thus, for embodiments where the light source is an LED, for example, LED error tolerance (e.g., +/−5 nm to +/−50 nm) can be mitigated to offer improvements to photon sensor 220 performance. Photon sensor 220 can be configured to generate an electrical signal in response to any wavelengths of light, and the short pass filter can facilitate control over what light reaches the photon sensor 220 and thus what the photon sensor 220 responds to. This also introduces flexibility in light source selection (e.g., in terms of emissions wavelengths).

Dichroic filter 240 allows for certain wavelengths to pass while blocking others. For example, in some embodiments, 450 nm wavelength light (and, e.g., other nearby wavelengths within a band) can be allowed to pass while blocking others. Dichroic filters can be configured to reflect away unwanted wavelengths based on the filter's configuration. Unwanted light can be reflected off a dichroic filter such that reflected rays leave the filter's surface at an angle relative to incident rays to minimize light interference. This effect can also be exploited to re-direct desirable light wavelengths.

Cut-on filter 252 can be configured as an inverse of a short pass optical filter. Thus, a cut-on filter can remove all wavelengths of light below a certain wavelength or range of wavelengths (e.g., the cut-on behavior is not perfectly binary) while allowing wavelengths above that cut-on wavelength to pass. Cut-on filter 252 thus further removes unwanted light before that light reaches second collimating optic 242 or photon sensor 220. By the time light reaches photon sensor 220, it should comprise predominantly a desired wavelength or set of wavelengths, which is then measured and interpreted. Thus, cut-on filter 252 wavelength values can be selected to establish a narrow range of wavelengths that are allowed to reach photon sensor 220. where light is able to pass through.

In some embodiments, through a combination of filtering and collimation, light that reaches photon sensor 220 is in the form of a beam comprised of electromagnetic radiation of a specific wavelength (or narrow band of wavelengths) with minor deviations from the nominal value when compared to the original light source. For example, if a 450 nm nominal value is implemented, the photon sensor's detectable wavelengths include are a range of values surrounding 450 nm. Thus, an LED could emit from 430 nm to 470 nm with varying intensity levels across that range with the most intense emissions at 450 nm though other wavelengths are also emitted between 430 nm and 470 nm, which could interfere with the quantification process if not handled appropriately. Filtering implemented into the system is intended to reduce the range of light reaching the photon sensor such that it is as close to the sensor's nominal value (in this case, 450 nm) as possible.

Although this application focuses on an embodiment of the inventive subject matter featuring two optically transparent measurement windows along with two light emitters (with corresponding hardware) in the testing device, additional measurement windows (e.g., 3+) can be included on test strips of the inventive subject matter, and a corresponding increase in the number of light emitters with corresponding hardware can be implemented into testing devices without deviating from the inventive subject matter.

While the examples described in this application are directed to using, e.g., a blood sample to measure phenylalanine levels in the blood, systems of the inventive subject matter should not be considered limited to phenylalanine detection. Instead, systems of the inventive subject matter can also detect other essential amino acids present in the blood. Because phenylalanine is one of nine essential amino acids, and because systems of the inventive subject matter are already expressly contemplated as being configured to detect phenylalanine, changes necessary to detect other amino acids involve adjustments to data interpretation as well as chemical reactants in test strips rather than hardware changes to testing devices.

FIG. 11 is a flowchart describing what happens with a blood sample when it is applied to a test strip of the inventive subject matter. First, whole blood is applied to a test strip where it undergoes plasma separation. Plasma separation can be accomplished as described above using a porous membrane to separate various blood cells and other matter from blood plasma. The plasma then undergoes a colorimetric reaction whereby non-light-absorbing residuals (e.g., chemicals that help to form the resulting dye that facilitates analyte quantification) are separated out from the plasma. Next, a dye is formed after a chemical reaction with the plasma (described above), and the dyed plasma is then ready for optical analysis. The dye is a byproduct of, e.g., a reaction the NADH component of blood plasma with tetrazolium salt, and the color of the dye can depend on that reaction. Conducting optical analysis results in output of an amino acid equivalent reading, which, can provide a user with, e.g., information critical to managing Phenylketonuria or other health-related information that can be determined by measuring amino acid concentrations in the blood.

Dye color is based on the type of salt. Example salts include: INT, MTT, XTT, MTS, and NBT. Once reacted with NADH, a formazan dye is created out of that reaction and the color depends on the salt involved in the reaction. Color also changes electromagnetic absorbance spectrum of the resulting solution. The formazan dye can thus be either soluble or non-soluble. Soluble versions flow better to the reaction site, while non-soluble versions precipitate out and get stuck. There are merits for both, and test strips can be designed to accommodate either or both variants.

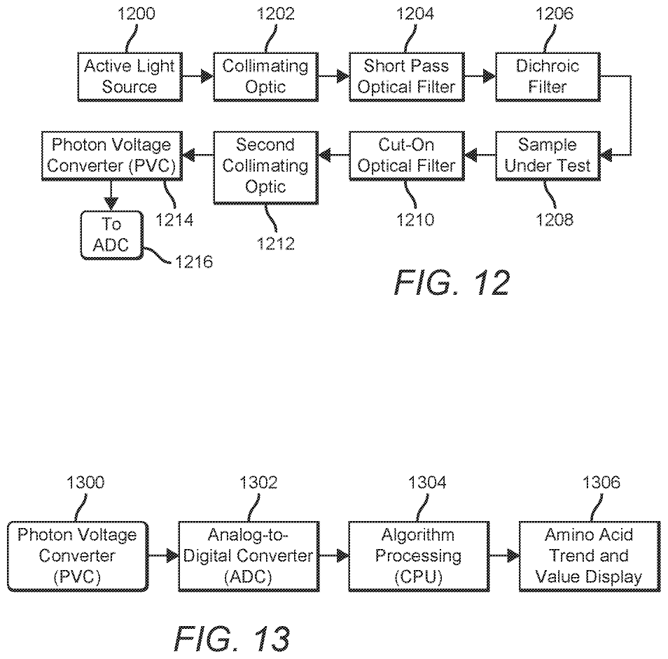

FIG. 12 is a flowchart describing the various functions of an optical module of a testing device of the inventive subject matter. The steps described in FIG. 12 correspond to the structural elements shown and described in FIG. 10. In step 1200, an optical module of the inventive subject matter is activated. Light emitted from that light source then passes through a collimating optic in step 1202. The collimating optic condenses that light into a largely beam configuration. In embodiments where the light source is a laser diode, the collimating optic can create a laser, but in instances where the light source is not a laser diode, collimating optic can still serve to form light passing through it largely into a beam configuration. Light from the collimating optic then passes through a short pass optical filter per step 1204 before next passing through dichroic filter in step 1206. In some embodiments the positions of the short pass filter and the dichroic filter can be reversed. In some embodiments, even the collimating optic can be repositioned into any order with respect to the short pass filter and the dichroic filter. Next, light passes through the sample under test in step 1208, where the sample under test is deposited on an optically transparent measurement window. From there, light passes through a cut-on optical filter per step 1210 before reaching a second collimating optic per step 1212. The second collimating optic again bring the light into a largely beam configuration to maximize the amount of light that has passed through the sample under test to reach the photon voltage converter in step 1214.

Photon voltage converter (PVC, also described above as a photon sensor) can be, e.g., a photodiode such as QSB34-D, a surface-mount photodiode. The exact photodiode used in embodiments of the inventive subject matter can vary based on analytical requirements, and it is contemplated that photodiodes of the inventive subject matter can be similar to, e.g., QSD2032-D, QSE773-D, and KFTEK-PM1125-WB, or any other silicon-based photodetector. Voltage data from the PVC is then transmitted to an analog-to-digital converter (ADC) where it is converted into a digital signal for processing and analysis. One example ADC is MCP3564R though many usable ADCs exist. MCP3564R is a Sigma-Delta style analog to digital converter, which offers high performance for low frequency signals. In some embodiments, the ADC is a separate component from the microcontroller, though it is contemplated that a system-on-chip (SOC) can be used that incorporates an ADC into the microcontroller.

FIG. 13 describes how information from a PVC becomes useful and readable information for a user. First, the PVC creates a voltage signal per step 1300, which can be conditioned to preserve signal integrity (e.g., scaled up or down by an amplifier, or filtered for specific signal frequencies). That voltage signal is then transmitted to an analog-to-digital converter per step 1302, and the ADC creates a digital signal from the analog voltage signal. These steps so far overlap with some of what has been described in FIG. 12. Next, the digital signal created by the ADC is transmitted to a CPU where it can be stored in memory in preparation for algorithmic processing. With the digital signal has been stored to CPU memory, the CPU can run a first algorithm to identify signatures within the data. After identifying certain data signatures with the first algorithm, software executed by the CPU can select a second algorithm with which to process the dataset to generate more information related to the previously identified data signatures, which can then be reported out as a set of values relating to those data signatures. For example, the second algorithm can calculate a measure of absorbance that a system of the inventive subject matter can then report to a user. Such a report to a user can include a concentration of phenylalanine within the sample once a calibration curve has been optimized for the system. An optimized calibration curve improves system sensitivity to measured changes in phenylalanine levels.

Selection of the second algorithm involves consideration for how intensely one or both of the light sources emits light. A calibration curve exists that maps light intensity to photon sensor interference. Light intensity mapping provides information to, e.g., the CPU such that once an initial reading is compared to an intensity map, the CPU can adjust current to the light source to match its output to an intensity mapping that correlates with more accurate readings. An intensity map can be selected such that the expected concentrations of an analyte is well understood. A calibration curve helps with an intensity mapping and makes it possible to adjust light source intensities to ensure those intensities are a range that produces accurate results regardless of analyte concentration thereby, increasing the dynamic range of detectable concentrations. When a different light source is implemented (e.g., a light source emitting a different wavelength or range of wavelengths of light), a different calibration curve is expected, which opens up the dynamic range of detectable concentrations.

A proprietary dynamic and intelligent estimation algorithm that is immune to background noise, can be implemented to assist in the determination of phenylalanine concentration levels. Raw data captured by the analog to digital converter will be stored into external memory (e.g., FRAM, FLASH, SRAM, etc.) from which the CPU can analyze the data by running such an algorithm to identify unique signatures within the data.

Each identified signature is composed of a set of values in the time and frequency domain, and each signature is also paired with a confidence level. Confidence level can be used to adjust LED intensity level, which leads to the generation of new data as a feedback mechanism for the algorithm to qualify signatures into the next processing stage. Once the confidence level exceeds a definable threshold, the algorithm will continue to process the raw data to quantify and report a phenylalanine concentration in a clear and concise fashion.

Calibration curves can be generated by conducting tests on samples having known concentrations of analyte. For example, a testing device can include LED light sources operating at a single intensity level for all sample analyte concentrations, and data collected by the testing device can then be fed into a single variable linear regression model according to Equation 1, below.

[Phe]=βₙAₘ(Iⱼ)+β₀ Equation 1

Where, [Phe], represents a concentration of phenylalanine (but can be, e.g., any other essential amino acid), βₙ represents a uniquely determined regression coefficient, Aₘ represents an absorption variable, Iⱼ represents current driving the LED light sources, and β₀ represents a constant offset term for the linear model. It has been observed that lower LED intensity levels result in improved performance when measuring samples having lower analyte concentrations, and higher LED intensities result in better performance when measuring samples having higher analyte concentrations. It has been discovered that by varying LED intensity, the dynamic range of the system can be expanded.

Equation 2, below, accommodates shifts in LED intensity levels, where Equation 2 is a function of independent absorption variable Aₘ and can represent, e.g., a super position of more than one linear model. Each Aₘ variable represents an absorption variable, and Iⱼ represents current through an LED emitting light at a known wavelength. The beta term (βₙ) is a uniquely determined regression coefficient, and β₀ is a constant offset term of the linear model. The absorption variable is a function of light source wavelength. For example, absorption at 450 nm will be different than at 470 nm. By conditioning a light source to emit light within a smaller range of wavelengths (e.g., +/−5 nm), error caused by absorption calculation can be limited.

In one example, current can be Iⱼ=150 μA, Iⱼ₊₁=400 μA, and Iⱼ₊₂=800 μA based on current passed to the light source within the testing device. reading from a testing device of the inventive subject matter at light source intensities set by an electrical current (e.g., 150 μA, 400 μA, and 800 μA).

[Phe]=βₙAₘ(Iⱼ)+βₙ₊₁Aₘ₊₁Aₘ₊₁(Iⱼ₊₁)+βₙ₊₂Aₘ₊₂(Iⱼ₊₂)+ . . . +β₀ Equation 2

Regarding detecting signatures in raw digital data, light sources in testing devices of the inventive subject matter can be modulated to have unique time domain-patterns. In some embodiments, light sources can additionally or alternatively be subjected to pulse width modulation to turn the lights sources on and back off at known frequencies and for known amounts of time Pulse width modulation offers advantages when looking at resulting waveforms generated by the photon sensor as such waveforms may not only look different from the original light source waveform, but patterns can also be discerned and correlated with analyte concentration. Since signal from a photon sensor is generated and captured over a set period of time, mathematical operations can be performed on this data to analyze the frequency components of the time domain signal (e.g., Fourier analysis), which can facilitate extracting unique signatures and patterns from the data.

FIG. 14 shows a cross section of each layer that can be included when creating a test strip of the inventive subject matter. Some layers may not be present in portions of the test strip, depending on desired functionality of that portion. The liquid sample insertion site, for example, comprises multiple layers to enable assay of phenylalanine using whole blood drops. Protection layers isolating chemical processes from a user are on top 1200 and bottom 1208. Top 1200 and bottom 1208 can also feature desirable optical properties, for example, 1200 can have optically transparent properties at a wavelength of interest. But bottom 1208 may have different optical properties; for example, bottom 1208 can be optically absorbent (e.g., it can prevent light from passing through it), in which case the location of a photon sensor can be changed such that it detects reflections from the liquid sample insertion site instead of transmissions through it. Bottom 1208 can also include an adhesive backing for support of additional layers or to be adhered to a rigid and reliable mechanical housing during use.

Plasma separation membrane 1202 filters out, e.g., red and white blood cells. As a blood passes through plasma separation membrane 1202, plasma is allowed to flow towards reaction sites supported by conjugate pad 1204 and nitrocellulose 1206 sub-layers. At the sample site side, this can be an asymmetric polysulfone material that, through capillary action, separates the red and white blood cells and allows plasma to pass through. In some embodiments, this material is located only on the sample site side. Conjugate pad 1204 can be made from, e.g., a glass fiber that will contain chemistry that mixes with a biological sample and travels to a next toward a nitrocellulose layer. A nitrocellulose membrane layer can contain additional chemicals to bring about a desired chemical reaction, and it may be treated with surfactant to improve flow performance. The structure of the liquid sample insertion site thus ensures that when blood is applied, plasma is allowed to flow to the optically transparent measurement windows via lateral flow pathways 408 and 410.

In some embodiments, nitrocellulose 1206 sub-layers can comprise an absorbent pad to facilitate better quantification performance. Such an absorbent pad can be made from a cotton linter material or cellulose, and the absorbent pad can absorb excess chemicals to prevent them from affecting the quantification process. If excess chemicals are not absorbed, those chemicals could, e.g., cause unwanted reflection or otherwise improperly mix with other layers or the biological sample in a way that negatively impacts testing performance.

Thus, specific systems and methods directed to testing devices with accompanying testing strips for the measurement of amino acid levels in blood have been disclosed. It should be apparent, however, to those skilled in the art that many more modifications besides those already described are possible without departing from the inventive concepts in this application. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. An analyte measurement system comprising:
a test strip, comprising:
a handling portion;
a liquid sample insertion site;
a lateral flow pathway connected to the liquid sample insertion site, the lateral flow pathway comprising a plasma separation membrane; and
an optically transparent measurement window coupled with the lateral flow pathway;
wherein the optically transparent measurement window comprises a reactant configured to create a dye when it reacts with a at least a portion of a liquid sample;
a testing device, comprising:
a cartridge port configured to receive the test strip;
a light source configured to emit light toward a photon sensor such that the optically transparent measurement window is disposed between the light source and the photon sensor when the test strip is inserted into the cartridge port;
an analog-to-digital converter (ADC) configured to convert an analog signal from the photon sensor into a digital signal; and
a microcontroller configured to determine an analyte concentration based on the digital signal.

2. The analyte measurement system of claim 1, wherein the reactant comprises a tetrazolium salt.

3. The analyte measurement system of claim 1, wherein the analyte comprises an amino acid.

4. The analyte measurement system of claim 1, wherein the amino acid comprises phenylalanine.

5. The analyte measurement system of claim 1, wherein the lateral flow pathway further comprises at least one of a nitrocellulose membrane, an asymmetric polysulfone, a hydrophilic glass fiber, and hydrophilic cotton linter materials.

6. The analyte measurement system of claim 1, wherein the liquid sample insertion site comprises a second plasma separation membrane layer, a conjugate pad layer, and a nitrocellulose sub-layer.

7. The analyte measurement system of claim 1, wherein the test strip further comprises a slot that is configured to ensure the test strip is seated and positioned properly upon insertion into the testing device.

8. The analyte measurement system of claim 1, wherein the light source emits light between 200 nm and 600 nm in wavelength.

9. A liquid biological sample testing device, comprising:
a cartridge port configured to receive a liquid biological sample test strip comprising an optically transparent measurement window;
a light source configured to emit light toward a photon sensor such that the optically transparent measurement window is disposed between the light source and the photon sensor when the liquid biological sample test strip is inserted into the cartridge port;
an analog-to-digital converter (ADC) configured to convert an analog signal from the photon sensor into a digital signal; and
a microcontroller configured to determine an analyte concentration in a liquid biological sample disposed on the liquid biological sample test strip based on the digital signal.

10. The testing device of claim 9, further comprising at least one of a collimating optic, a short pass optical filter, a dichroic filter, and a cut-on optical filter.

11. The testing device of claim 9, wherein the light source emits light between 200 nm and 600 nm in wavelength.

12. A liquid biological sample test strip, comprising:
a handling portion;
a liquid sample insertion site;
a lateral flow pathway connected to the liquid sample insertion site, the lateral flow pathway comprising a plasma separation membrane;
an optically transparent measurement window coupled with the lateral flow pathway;
wherein the optically transparent measurement window comprises a reactant configured to create a dye when it reacts with at least a portion of a liquid sample; and
a slot that is configured to ensure the test strip is seated and positioned properly upon insertion into a testing device.

13. The test strip of claim 12, wherein the lateral flow pathway further comprises at least one of a nitrocellulose membrane, a hydrophilic glass fiber, and hydrophilic cotton linter materials.

14. The test strip of claim 12, wherein the reactant comprises a tetrazolium salt.

15. The test strip of claim 12, wherein the liquid sample insertion site comprises a second plasma separation membrane layer, an asymmetric polysulfone, a conjugate pad layer, and a nitrocellulose sub-layer.

16. The test strip of claim 12, further comprising a slot that is configured to ensure the test strip is seated and positioned properly upon insertion into the testing device.
